(19) United States
(12) Patent Application Publication
(10) Pub. No.: US 2011/0168200 A1
(43) Pub. Date: Jul. 14, 2011

(54) COSMETIC COMPOSITION COMPRISING AT LEAST ONE TYPE OF HOLLOW PARTICLE, AT LEAST ONE STARCH AND AT LEAST ONE CATIONIC SURFACTANT

(76) Inventors: Claire BOURDIN, Levallois-Perret (FR); Céline Sellier, Paris (FR); Gérard Gabin, Paris (FR)

(21) Appl. No.: 12/946,098

(22) Filed: Nov. 15, 2010

Related U.S. Application Data
(60) Provisional application No. 61/264,292, filed on Nov. 25, 2009.

(30) Foreign Application Priority Data
Nov. 13, 2009 (FR) 0958026

(51) Int. Cl. A61K 8/02; A61K 8/73; A61Q 5/12

(52) U.S. Cl. 132/202; 424/401; 424/70.27

(57) ABSTRACT

The present disclosure relates to cosmetic compositions in the form of a deformable solid comprising, in a cosmetically acceptable medium, at least one type of hollow particle, at least one starch chosen from modified and unmodified starches, and at least one cationic surfactant, wherein the weight ratio of the at least one cationic surfactant to at least one starch is greater than or equal to 0.3.

This application claims benefit of U.S. Provisional Application No. 61/264,292, filed Nov. 25, 2009. This application also claims benefit of priority under 35 U.S.C. §119 to French Patent Application No. 0958026, filed Nov. 13, 2009.

The present disclosure relates to cosmetic compositions in the form of a deformable solid comprising hollow particles, at least one modified or unmodified starch and at least one cationic surfactant, with a particular ratio of at least one starch to at least one cationic surfactant.

Numerous hair care products that impart good cosmetic properties to the hair are known in the art.

The conventional products for hair care, for example after-shampoo products or masks, are most often in the form of more or less viscous creams. These fluid products are difficult to measure out, for example because they tend to slip away between the fingers or leak from their packaging, which can be very troublesome when they come in contact with clothing, for instance when travelling.

Adding thickeners or increasing the proportion of oily phase to conventional products may modify the texture and notably make it denser. However, this is often to the detriment of the cosmetic effects of the composition.

Moreover, the thickest conditioning compositions may often have the drawback of possibly requiring a lot of rinsing water to remove the surplus of product on the hair. In many countries where access to water is restricted, the rinsing time and therefore the amount required for proper rinsing of the product are key indicators of the utility qualities of a composition.

Moreover, hair care compositions, whether or not they are products requiring rinsing, constitute for the most part suspensions of crystals of fats. These suspensions do not always allow the desired raw materials to be incorporated, or incorporated in the desired proportion, which may lead to destabilization effects.

Finally, users are forever looking for new textures and new types of products.

Thus, there is a need for hair care compositions that do not flow and that are denser, that require little water for rinsing and that make it possible to avoid phenomena of aggregation of crystals and therefore flocculation. The required compositions must be easy to apply on the hair, and they must be very light and very smooth.

The present disclosure therefore relates to compositions comprising a combination of hollow particles, at least one starch and at least one cationic surfactant, with a particular ratio of at least one starch to at least one cationic surfactant, which may make it possible to solve at least one of the problems of the prior art.

Thus, one aspect of the present disclosure relates to a cosmetic composition comprising, in a cosmetically acceptable medium, at least one type of hollow particle, at least one starch chosen from modified and unmodified starches, and at least one cationic surfactant, wherein the weight ratio of the at least one cationic surfactant to the at least one starch has a value greater than or equal to 0.3, and wherein the cosmetic composition is in the form of a deformable solid.

As used herein, “cosmetic composition in the form of a deformable solid” is understood to mean a composition that is in a solid, dry, malleable form, resembling marshmallow (see, for example, U.S. Pat. No. 3,682,659 for the consistency of marshmallow).

The specific gravity of the composition according to the present disclosure ranges from 0.2 to 0.6, such as ranges from 0.4 to 0.5.

The compositions according to the present disclosure may help prevent the risk of it leaking from its packaging, notably during transport. Moreover, the compositions disclosed herein are very easy to take hold of, do not flow away between the fingers, and can be much easier to measure out than the liquid compositions.

The compositions according to the present disclosure may also exhibit good properties of melting or disintegrating during the application. The hair is supple at the time of application. The compositions disclosed herein may also lead to properties of suppleness of the hair on rinsing, of disentangling and of suppleness on wet hair, and of smoothing on dry hair. Moreover, the compositions disclosed herein may exhibit good disintegration and result in a smooth texture after being converted to a cream in the hand.

The compositions according to the present disclosure may also exhibit excellent comfort during application. For example, the compositions are capable of not running at all, in contrast to conventional compositions, where there is a risk of irritation, notably of the face and eyes. The absence of running is greatly appreciated in the case of perms and dyes, as well as for shampoos intended for children.

According to the present disclosure, the compositions can be in the form of deformable solids that are capable of not staining and resemble marshmallows. These solids can be modelled, for example like modelling clay for children. They can be easily broken by hand so as to take only the required amount of product. For instance, these compositions can be packaged in monodose form, for example in the form of small cubes or pieces. From a bacteriological standpoint, packaging in monodose form is beneficial because the product is consumed completely in a single application and there is no remaining product that could be contaminated after contact with the hands and/or the surrounding air. This means that it is possible to use less concentrated preservative systems.

The hollow particles may allow the compositions of the present disclosure to have a homogeneous structure (deformable solid) comprising constituents that normally lead to separate phases (immiscible constituents, for example oil and water).

In at least one embodiment of the present disclosure, the hollow particles are chosen from glass particles, particles of thermoplastic materials chosen from polyamides such as nylon, polymers and copolymers of acrylonitrile, particles of vinylidene chloride, particles of vinyl chloride, particles of acrylic monomer, particles of styrene monomer, all optionally expanded, microporous microspheres and silica microparticles.

The acrylic monomer is, in at least one embodiment, chosen from methyl acrylate, ethyl acrylate and methacrylate. The styrene monomer is for example chosen from α-methylstyrene and styrene.

Suitable glass particles according to the present disclosure include for example, the hollow glass beads sold by the company 3M under the reference SCOTCHLITE GLASS BUBBLES S 22. 95% of these beads have a diameter below 74 μm.

According to at least one embodiment of the present disclosure, the hollow particles are deformable hollow particles of an expanded copolymer of vinylidene chloride and acrylonitrile, or of vinylidene chloride, acrylonitrile and (meth)acrylate or styrene monomer. It is possible for example to use a polymer containing 0-60% of units derived from vinylidene chloride, 20-90% of units derived from acrylonitrile and 0-50% of units derived from an acrylic or styrene monomer, the sum of the percentages (by weight) being equal to 100%, wherein the acrylic monomer can be chosen from methyl, ethyl and (meth)acrylate. The styrene monomer can be chosen from styrene and α-methylstyrene.

In another embodiment of the present disclosure, the hollow particles are hollow particles of an expanded copolymer of vinylidene chloride and acrylonitrile or of vinylidene chloride, acrylonitrile and methyl methacrylate. These particles can be dry or hydrated.

In yet another embodiment, the hollow particles are, for example, chosen from microspheres of expanded terpolymer of vinylidene chloride, acrylonitrile and methyl methacrylate, sold under the brand name EXPANCEL by the company Nobel Casco, such as under the references 551 DE 12 (granulometry D(0.5) of about 12 μm and density of about 40 kg/m³), 551 DE 20 (granulometry D(0.5) of about 15 to 25 μm and density of about 60 kg/m³), 551 DE 50 (granulometry D(0.5) of about 40 μm), 461 DE 50 and 642 WE 50 of 50 μm with granulometry of about D(0.5), 551 DE 80 (granulometry D(0.5) from about 50 to 80 μm). It is also possible to use hollow particles of this same expanded terpolymer, having a granulometry D(0.5) of about 18 μm and a density of about 60 to 80 kg/m³ (EXPANCEL EL23) or with a granulometry D(0.5) of about 34 μm and density of about 20 kg/m³. Other suitable examples include, but are not limited to the particles EXPANCEL 551 DE 40 d42 (granulometry D(0.5) of about 30 to 50 μm and density of about 42 kg/m³), 551 DE 80 d42 (granulometry D(0.5) of about 50 to 80 μm and density of about 42 kg/m³), 461 DE 20 d70 (granulometry D(0.5) of about 15 to 25 μm and density of about 70 kg/m³), 461 DE 40 d25 (granulometry D(0.5) of about 35 to 55 μm and density of about 25 kg/m³), 461 DE 40 d60 (granulometry D(0.5) of about 20 to 40 μm and density of about 60 kg/m³), 461 DET 40 d25 (granulometry D(0.5) of about 35 to 55 μm and density of about 25 kg/m³), 051 DE 40 d60 (granulometry D(0.5) of about 20 to 40 μm and density of about 60 kg/m³), 091 DE 40 d30 (granulometry D(0.5) of about 35 to 55 μm and density of about 30 kg/m³), 091 DE 80 d30 (granulometry D(0.5) of about 60 to 90 μm and density of about 30 kg/m³). It is also possible to use hollow particles of unexpanded polymer of vinylidene chloride and acrylonitrile or of vinylidene chloride, acrylonitrile and methyl methacrylate such as those sold under the brand name EXPANCEL with the reference 551 DU 10 (granulometry D(0.5) of about 10 μm) or 461 DU 15 (granulometry D(0.5) of about 15 μm).

Non-limiting mention may also be made of microporous microspheres sold by Dow Corning under the designation “POLYTRAP” which are formed from lauryl methacrylate/ethylene glycol dimethacrylate copolymers; or those sold by Seppic under the designation “MICROPEARL”.

Suitable silica microparticles according to the present disclosure, include, but are not limited to hollow silica microparticles such as those sold by Miyoshi Kasei under the designation “SILICA BEADS S700”.

Other examples of hollow polymer particles that may be used in the compositions of the present disclosure include polymers and copolymers obtained from itaconic, citraconic, maleic, fumaric esters or acids, from vinyl acetate or lactate.

In at least one embodiment, the density of the hollow particles according to the present disclosure ranges from 15 to 200 kg/m³, for example, ranges from 20 to 120 kg/m³, such as ranges from 30 to 80 kg/m³.

The hollow particles present in the compositions according to the present disclosure generally have a mean granulometry D(0.5) ranging from 1 μm to 300 μm, for example ranging from 5 μm to 200 μm, such as ranging from 10 μm to 100 μm, for instance ranging from 15 μm to 50 μm.

The hollow particles present in the compositions according to at least one embodiment are filled with at least one gas chosen from air, nitrogen, isobutane and isopentane.

The hollow particles in at least one embodiment of the present disclosure are chosen from microspheres of expanded terpolymer of vinylidene chloride, acrylonitrile and methyl methacrylate.

The hollow particles can be present in the compositions disclosed herein in an amount ranging from 0.01 to 15 weight. %, such as ranging from 1 to 10 weight. %, relative to the total weight of the composition.

The compositions according to the present disclosure comprise at least one starch chosen from modified and unmodified starches. For example, the at least one starch chosen from modified and unmodified starches can be chosen from macromolecules in the form of polymers constituted of elementary units that are anhydroglucose units. The number of these units and their assembly make it possible to distinguish between amylose (linear polymer) and amylopectin (branched polymer). The relative proportions of amylose and amylopectin, as well as their degree of polymerization, vary depending on the vegetable origin of the starches.

The at least one starch can, for example, be obtained from a vegetable source such as cereals, tubers, roots, vegetables and fruits. Thus, the at least one starch can be obtained from a vegetable source chosen from maize, peas, potato, sweet potato, banana, barley, wheat, rice, oat, sago, tapioca and sorghum. According to at least one embodiment, the at least one starch is obtained from potato.

The hydrolysates of the at least one starch can also be used.

The at least one starch can be, for example in the form of a white powder, insoluble in cold water, with the elementary particles ranging in size from 3 to 100 microns.

The at least one starch used in the compositions disclosed herein can be modified chemically by at least one of the following reactions: pregelatinization, oxidation, crosslinking, esterification, and thermal treatments.

According to at least one embodiment of the present disclosure, these reactions can be carried out as follows:

pregelatinization by making the starch grains burst (for example drying and cooking in a drying drum);

oxidation by strong oxidizing agents leading to introduction of carboxyl groups in the starch molecule and to depolymerization of the starch molecule (for example by treating an aqueous solution of starch with sodium hypochlorite);

crosslinking by functional agents capable of reacting with the hydroxyl groups of the starch molecules which will thus be joined together (for example with glyceryl and/or phosphate groups);

esterification in an alkaline medium for grafting functional groups, for instance C₁-C₆ acyl (acetyl), C₁-C₆ hydroxyalkyl (hydroxyethyl, hydroxypropyl), carboxyalkyl (for instance carboxymethyl), octenylsuccinic. According to at least one embodiment, the at least one starch is modified with carboxymethyl sodium.

Other suitable examples of the at least one starch include monostarch phosphates (of the type Am—O—PO—(OX)2), distarch phosphates (of the type Am—O—PO—(OX)—O—Am), tristarch phosphates (of the type Am—O—PO—(O—Am)2), and mixtures thereof, which are obtained by crosslinking with phosphorus-containing compounds; wherein X is chosen from alkali metals (for example sodium or potassium), alkaline-earth metals (for example calcium, magnesium), salts of ammonia, and salts of amines such as those of monoethanolamine, diethanolamine, triethanolamine, and amino-3-propanediol-1,2, ammonium salts derived from basic amino acids such as lysine, arginine, sarcosine, ornithine, and citrulline.

The phosphorus-containing compounds can be chosen from, for example sodium tripolyphosphate, sodium orthophosphate, phosphorus oxychloride and sodium trimetaphosphate.

In at least one embodiment, distarch phosphates chosen from hydroxypropylated starches, are used in the compositions disclosed herein, such as the product offered under the references PREJEL VA-70-T AGGL (gelatinized hydroxypropylated manioc distarch phosphate) or PREJEL TK1 (gelatinized manioc distarch phosphate) or PREJEL 200 (gelatinized acetylated manioc distarch phosphate) by the company AVEBE or STRUCTURE ZEA from NATIONAL STARCH (gelatinized hydroxypropylated maize distarch phosphate).

When the starches are modified chemically by an esterification reaction, it is possible to obtain carboxyalkylstarches.

In at least one embodiment of the present disclosure, the carboxyalkylstarches are chosen from carboxyalkyl (C₁-C₄) starches, such as carboxymethylstarches.

The salts are for instance alkali metal or alkaline-earth metal salts such as Na, K 1/2, Li, NH₄, of a quaternary ammonium or of an organic amine such as mono-, di- or triethanolamine.

The carboxyalkylstarches are obtained by grafting carboxyalkyl groups on at least one alcohol function of starch, such as by reaction of starch and sodium monochloroacetate in an alkaline medium.

The carboxyalkyl groups are, for example, fixed via an ether function, such as on carbon 1.

The degree of substitution is for instance from 0.1 to 1, such as from 0.15 to 0.5. As used herein, the degree of substitution is understood to mean the average number of hydroxyl groups substituted with an ester or ether group (in the present case ether for the carboxymethylstarches) per monosaccharide unit of the polysaccharide.

Suitable carboxyalkylstarches according to the present disclosure comprise units of the following formula:

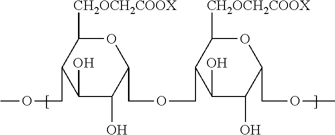

wherein X is chosen from a hydrogen atom, alkali metals, and alkaline-earth metals such as Na, K ½, Li, NH₄, quaternary ammonium and organic amines. In at least one embodiment, X is an Na+ ion.

The carboxyalkylstarches in at least one embodiment of the present disclosure are non-pregelatinized carboxyalkylstarches.

In another embodiment of the present disclosure, the carboxyalkylstarches are chosen from partially and fully crosslinked carboxyalkylstarches.

Other suitable carboxyalkylstarches include, but are not limited to sodium salts of carboxyalkylstarches, for example sodium salt of potato carboxymethylstarch sold under the designation PRIMOJEL by the company DMV International. More than 95% of the particles of this starch have a diameter below 100 microns, such as below 65 microns.

According to at least one embodiment of the present disclosure, it is also possible to use amphoteric starches comprising at least one anionic group and at least one cationic group. The anionic and cationic groups can be bound to the same reactive site of the starch molecule or to different reactive sites, and in at least one embodiment, they are bound to the same reactive site. The anionic groups can be chosen from carboxyl, phosphate and sulphate anionic groups, for example carboxyl. The cationic groups can be chosen from primary, secondary, tertiary and quaternary amines.

Suitable amphoteric starches can be chosen, for example, from the compounds of the following formulae:

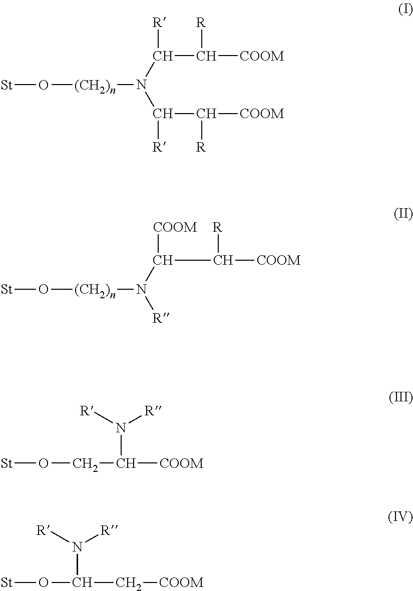

wherein

St-O is a starch molecule,

Each instance of R is independently chosen from a hydrogen atom and a methyl radical,

Each instance of R′ is independently chosen from a hydrogen atom, methyl radicals, and —COOH,

n is an integer equal to 2 or 3,

Each instance of M is independently chosen from a hydrogen atom, alkali metals, alkaline-earth metals such as Na, K, Li, NH₄, a quaternary ammonium and an organic amine,

R″ is chosen from a hydrogen atom and C₁-C₁₈ alkyl radicals.

Such compounds are described, for instance in U.S. Pat. Nos. 5,455,340 and 4,017,460.

According to at least one embodiment, the at least one starch is chosen from compounds of formulae (I) and (II), for example starches modified with 2-chloroethyl aminodipropionic acid, wherein R, R′, R″ and M are hydrogen atoms and n is 2. For instance, potato starch modified with 2-chloroethyl aminodipropionic acid neutralized with soda, marketed under the reference STRUCTURE SOLANACE by the company NATIONAL STARCH, may be used in the compositions disclosed herein.

In at another embodiment of the present disclosure, the at least one starch is chemically modified.

The at least one starch present in the compositions according to the present disclosure are present in an amount ranging from 0.01 to 15 weight. %, for instance ranging from 0.1 to 10 weight. %, such as ranging from 1 to 10 weight. %, relative to the total weight of the composition.

According to the present disclosure, the at least one cationic surfactant may be chosen, by way of non-limiting example, from salts of primary, secondary and tertiary fatty amines, all optionally polyoxyalkylated; quaternary ammonium salts such as tetraalkylammonium, alkylamidoalkyltrialkylammonium, trialkylbenzylammonium, trialkylhydroxyalkylammonium, alkylpyridinium chlorides and alkylpyridinium bromides; imidazoline derivatives; and cationic amine oxides.

Non-limiting mention may be made of methyl alkyl alkylamidoethyl imidazolinium methosulphate (Quaternium-87) marketed, for instance by the company DEGUSSA under the reference VARISOFT W 575 PG, or the behenyl trimethyl ammonium chloride marketed, for example by the company CLARIANT under the reference GENAMIN KDMP, and cetyl trimethyl ammonium chloride.

Other suitable examples of the at least one cationic surfactant include, but are not limited to quaternary ammonium salts containing at least one ester function such as those of the following formula (V):

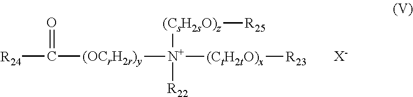

wherein:

R₂₂ is chosen from linear and branched C₁-C₆ alkyl radicals, C₁-C₆ hydroxyalkyl radicals, and C₁-C₆ dihydroxyalkyl radicals;

R₂₃ is chosen from:
the radical

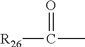

linear and branched, saturated and unsaturated, C₁-C₂₂ hydrocarbon radicals R₂₇, and
a hydrogen atom,
R₂₅ is chosen from:
the radical

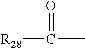

linear and branched, saturated and unsaturated C₁-C₆ hydrocarbon radicals R₂₉, and
a hydrogen atom,

R₂₄, R₂₆ and R₂₈, are each independently chosen from linear and branched, saturated and unsaturated C₇-C₂₁ hydrocarbon radicals;

r, s and t, are each independently chosen from integers ranging from 2 to 6;

y is an integer ranging from 1 to 10;

x and z, are each independently chosen from integers ranging from 0 to 10;

X⁻ is a simple or complex, organic or inorganic anion;
provided that the sum x+y+z has a value ranging from 1 to 15, and that when x has the value 0, R₂₃ is R₂₇, and that when z has the value 0, R₂₅ is R₂₉.

According to at least one embodiment of the present disclosure, the alkyl radicals R₂₂ are linear.

In another embodiment of the present disclosure, R₂₂ is chosen from methyl, ethyl, hydroxyethyl and dihydroxypropyl radicals, and in at least one embodiment, chosen from methyl and ethyl radicals.

According to at least one embodiment of the present disclosure, the sum x+y+z has a value ranging from 1 to 10.

When R₂₃ is a hydrocarbon radical R₂₇, it can be long and comprise 12 to 22 carbon atoms, or short and comprise 1 to 3 carbon atoms.

When R₂₅ is a hydrocarbon radical R₂₉, in at least one embodiment, R₂₅ comprises 1 to 3 carbon atoms.

In at least one embodiment of the present disclosure, R₂₄, R₂₆ and R₂₈, are each independently chosen from linear and branched, saturated and unsaturated C₁₁-C₂₁ hydrocarbon radicals, for example chosen from linear and branched, saturated and unsaturated C₁₁-C₂₁ alkyl and alkenyl radicals.

According to at least one embodiment, x and z, each independently have the value 0 or 1, for example y is 1.

In at least one embodiment, r, s and t, each independently have the value 2 or 3, for example r, s and t each have the value 2.

The anion is for example a halide chosen from chloride, bromide and iodide; or an alkylsulphate, such as methylsulphate. Mention may also be made, in a non-limiting manner, of methanesulphonate, phosphate, nitrate, tosylate, anions derived from organic acids such as acetate, lactate and any other anion compatible with the ammonium with ester function.

According to at least one embodiment, the anion X″ is chosen from chloride and methylsulphate.

Suitable examples of ammonium salts of formula (V)), include, but are not limited to compounds wherein:
R₂₂ is chosen from methyl and ethyl radicals,
x and y are equal to 1;
z is equal to 0 or 1;
r, s and t are equal to 2;
R₂₃ is chosen from:
the radical

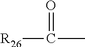

methyl, ethyl and C₁₄-C₂₂ hydrocarbon radicals, and
a hydrogen atom;
R₂₅ is chosen from:
the radical

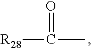

and
a hydrogen atom; and
R₂₄, R₂₆ and R₂₈, are each independently chosen from linear and branched, saturated and unsaturated C₁₃-C₁₇ hydrocarbon radicals, for example, linear and branched, saturated and unsaturated C₁₃-C₁₇ alkyl and alkenyl radicals.

According to at least one embodiment of the present disclosure, the hydrocarbon radicals are linear.

Non-limiting mention may be made for example, of the compounds of formula (V) such as the diacyloxyethyl-dimethylammonium, diacyloxyethyl-hydroxyethyl-methylammonium, monoacyloxyethyl-dihydroxyethyl-methylammonium, triacyloxy ethyl-methylammonium, monoacyloxyethyl-hydroxyethyl-dimethyl ammonium salts (for instance chloride or methylsulphate) and mixtures thereof. The acyl radicals can have, for instance 14 to 18 carbon atoms and can be derived from a vegetable oil such as palm oil or sunflower oil. When the compound contains several acyl radicals, the latter can be identical or different. Mention may also be made, for instance of distearoylethyl hydroxyethylammonium methosulphate.

These products are obtained, for example, by direct esterification of triethanolamine, triisopropanolamine, alkyldiethanolamine or alkyldiisopropanolamine optionally oxyalkylated on fatty acids or on mixtures of fatty acids of vegetable or animal origin, or by transesterification of their methyl esters. This esterification is followed by quaternization using an alkylating agent such as an alkyl (for instance methyl or ethyl) halide, a dialkyl (preferably methyl or ethyl) sulphate, methyl methanesulphonate, methyl paratoluenesulphonate, glycol or glycerol chlorohydrin.

Such compounds are for instance marketed under the designations DEHYQUART® by the company HENKEL, STEPANQUAT® by the company STEPAN, NOXAMIUM® by the company CECA, REWOQUAT® WE 18 by the company REWO-WITCO.

The compositions according to the present disclosure can, for example, contain a mixture of quaternary ammonium mono-, di- and triester salts with a majority by weight of diester salts.

As the mixture of ammonium salts, it is possible to use for example a mixture containing 15 to 30 weight. % of acyloxyethyl-dihydroxyethyl-methylammonium methylsulphate, 45 to 60% of diacyloxyethyl-hydroxyethyl-methylammonium methylsulphate and 15 to 30% of triacyloxyethyl-methylammonium methylsulphate, the acyl radicals having from 14 to 18 carbon atoms and being derived from palm oil, optionally partially hydrogenated.

It is also possible to use the ammonium salts containing at least one ester function, such as the salts described in U.S. Pat. Nos. 4,874,554 and 4,137,180.

According to the present disclosure, the at least one cationic surfactant is present in an amount ranging from 0.1 to 10 weight. % relative to the total weight of the composition, and the weight ratio of the at least one cationic surfactant to at least one starch has a value greater than or equal to 0.3. In at least one embodiment of the present disclosure, the weight ratio of the at least one cationic surfactant to at least one starch has a value ranging from 0.3 to 10, such as from 0.35 to 5, for example from 0.35 to 1.

The compositions according to the present disclosure can also comprise at least one additional particulate agent different from the hollow particles described above.

The at least one additional particulate agent is, for example, smaller than 400 and can be of natural or synthetic origin.

The at least one additional particulate agent is chosen from vegetable powders, pigments and anti-dandruff agents.

The pigments can be chosen from organic or mineral pigments.

Non-limiting examples of mineral pigments include, but are not limited to titanium dioxide (rutile or anatase) optionally surface-treated and classified in the Color Index under the reference CI 77891; black, yellow red and brown iron oxides, classified under references CI 77499, 77492, 77491; manganese violet (CI 77742); ultramarine (CI 77007); hydrated chromium oxide (CI 77289); ferric blue (CI 77510).

Suitable organic pigments include, but are not limited to pigment YELLOW 3 notably sold under the trade name “YELLOW COVANOR W 1603” by the company WACKHERR (CI 17710), “D & C RED No. 19” (CI 45170), “D & C RED No. 9 (CI 15585), “D & C RED No 21 (CI 45380), “D & C ORANGE No 4” (CI 15510), “D & C ORANGE No 5” (CI 45370), “D & C RED No 27” (CI 45410), “D & C RED No 13” (CI 15630), “D & C RED No 7” (CI 15850-1), “D & C RED No 6” (CI 15850-2), “D & C YELLOW No 5” (CI 19140), “D & C RED 36” (CI 12085), “D & C ORANGE No 10” (CI 45425), “D & C YELLOW No. 6” (CI 15985), “D & C RED No. 30” (CI 73360), “D & C RED No. 3” (CI 45430), “D & C RED No. 4” (CI 14700), carbon black (CI 77266), and lakes based on carmine (CI 75470).

It is also possible to use nacreous pigments chosen from white nacreous pigments such as mica covered with titanium oxide, bismuth oxide; coloured nacreous pigments such as titanium mica with iron oxides, titanium mica with ferric blue or chromium oxide, titanium mica with an organic pigment of the precipitated type, as well as those based on bismuth oxychloride.

Non-limiting mention may also be made of pigment pastes of organic pigment such as the products sold by the company HOECHST under the names:
JAUNE COSMENYL 10G: Pigment YELLOW 3 (CI 11710)
JAUNE COSMENYL G: Pigment YELLOW 1 (CI 11680)
ORANGE COSMENYL GR: Pigment ORANGE 43 (CI 71105)
ROUGE COSMENYL R°: Pigment RED 4 (CI 12085)
CARMIN COSMENYL FB: Pigment RED 5 (CI 12490)
VIOLET COSMENYL RL: Pigment VIOLET 23 (CI 51319)
BLEU COSMENYL A2R: Pigment BLUE 15.1 (CI 74260)
VERT COSMENYL GG: Pigment GREEN 7 (CI 74260)
NOIR COSMENYL R: Pigment BLACK 7 (CI 77266)

Suitable vegetable powders include, but are not limited to spruce sawdust (extract of Picea excelsa) and powder of ground apricot stones, for example with a granulometry ranging from 300 μm to 400 μm.

In at least one embodiment of the present disclosure, the at least one additional particulate agent different from the hollow particles present in the compositions disclosed herein are present in an amount ranging from 0.01 to 30 weight. % relative to the total weight of the composition.

The compositions according to the present disclosure can further comprise at least one non-silicone fat.

In at least one embodiment, the at least one non-silicone fat is chosen from fatty alcohols, fatty acids, esters of fatty acid and of fatty alcohol, waxes, vegetable, animal, mineral and synthetic oils.

Exemplary fatty alcohols include, but are not limited to alcohols of formula R′OH, wherein R′ is chosen from saturated and unsaturated, linear and branched radicals, such as C₈-C₄₀ radicals, for instance, C₈-C₃₀ radicals. In at least one embodiment, R′ is chosen from C₁₂-C₂₄ alkyl groups and C₁₂-C₂₄ alkenyl groups, and is optionally substituted with at least one hydroxyl group.

Other suitable examples of the at least one fatty alcohol include, but are not limited to lauric alcohol, cetyl alcohol, dodecyl alcohol, decyl alcohol, stearyl alcohol, oleic alcohol, behenic alcohol, linoleic alcohol, undecylenic alcohol, palmitoleic alcohol, arachidonic alcohol, myristyl alcohol and erucic alcohol, and mixtures thereof. Non-limiting examples of mixtures of fatty alcohols include cetylstearyl and cetearyl alcohol.

In accordance with the present disclosure, the at least one fatty acid can be chosen from acids of formula RCOOH, wherein R is chosen from saturated and unsaturated, linear and branched radicals, such as C₇-C₃₉ radicals.

In at least one embodiment, R is chosen from C₇-C₂₉ alkyl groups and C₇-C₂₉ alkenyl groups, such as C₁₂-C₂₄ alkyl groups and C₁₂-C₂₄ alkenyl groups. In at least one embodiment, R can be substituted with at least one hydroxyl group and/or at least one carboxyl group.

Esters of fatty acid can be chosen, for example, from lauric acid, oleic acid, palmitic acid, linoleic acid, myristic acid and stearic acid.

Suitable vegetable oils include, but are not limited to jojoba oil, avocado oil, colza oil, olive oil, sunflower oil, maize oil, soya oil, cucurbit oil, grapeseed oil, sesame oil, hazelnut oil, apricot oil, macadamia oil, arara oil, and castor oil.

The at least one non-silicone fat in the compositions disclosed herein may be present in an amount ranging from 0.5 to 20 weight. % relative to the total weight of the composition.

The compositions according to the present disclosure can also comprise at least one silicone.

The silicones optionally present in the compositions according to the present disclosure are, for example, polyorganosiloxanes, which can be in the form of aqueous solutions such as solubilized solutions, or optionally in the form of dispersions or micro-dispersions, or aqueous emulsions. The polyorganosiloxanes disclosed herein can also be in the form of oils, waxes, resins or gums.

The organopolysiloxanes are, for instance, defined in more detail in the work by Walter NOLL “Chemistry and Technology of Silicones” (1968) Academic Press.

The silicones can be volatile or non-volatile.

When they are volatile, the silicones are, for example, chosen from silicones having a boiling point ranging from 60° C. to 260° C. Suitable examples include, but are not limited to:

(i) cyclic silicones having from 3 to 7 silicon atoms, for example from 4 to 5 silicon atoms, such as the octamethylcyclotetrasiloxane marketed under the name “VOLATILE SILICONE 7207” by UNION CARBIDE and “SILBIONE 70045 V 2” by RHONE POULENC, decamethylcyclopentasiloxane marketed under the name “VOLATILE SILICONE 7158” by UNION CARBIDE, “SILBIONE 70045 V 5” by RHONE POULENC, and mixtures thereof.

Non-limiting mention may also be made of cyclocopolymers of the dimethylsiloxanes/methylalkylsiloxane type, such as “SILICONE VOLATILE FZ 3109” marketed by the company UNION CARBIDE, of chemical structure:

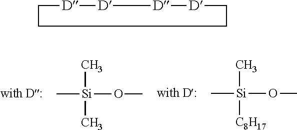

Non-limiting mention may also be made of mixtures of cyclic silicones with organic compounds derived from silicon, such as the mixture of octamethylcyclotetrasiloxane and tetratrimethylsilylpentaerythritol (50/50) and the mixture of octamethylcyclotetrasiloxane and of oxy-1,1′(hexa-2,2,2′,2′,3,3′-trimethylsilyloxy)bis-neopentane;

(ii) linear volatile silicones having 2 to 9 silicon atoms and having a viscosity less than or equal to 5.10⁻⁶ m²/s at 25° C., for example, decamethyltetrasiloxane marketed under the designation “SH 200” by the company TORAY SILICONE. Other suitable examples of such silicones also include, but are not limited to those described in the article published in Cosmetics and toiletries, Vol. 91, Jan. 76, p. 27-32—TODD & BYERS “Volatile Silicone fluids for cosmetics”.

According to at least one embodiment of the present disclosure, non-volatile silicones chosen from polyalkylsiloxanes, polyarylsiloxanes, polyalkylarylsiloxanes, silicone gums and resins, polyorganosiloxanes modified with organofunctional groups, and mixtures thereof are used in the compositions of the present disclosure.

Exemplary silicones include, but are not limited to polyalkylsiloxanes such as polydimethylsiloxanes with trimethylsilyl end groups (Dimethicone according to the CTFA designation) having a viscosity ranging from 5.10⁻⁶ to 2.5 m²/s at 25° C., such as ranging from 1.10⁻⁵ to 1 m²/s. The viscosity of the silicones is for example measured at 25° C. according to standard ASTM 445 Appendix C.

Among those polyalkylsiloxanes, non-limiting mention may be made of the following commercial products:
the SILBIONE oils of series 47 and 70 047 or the MIRASIL oils marketed by RHONE POULENC, for example oil 70 047 V 500 000;
oils of the MIRASIL series marketed by the company RHONE POULENC;
oils of the 200 series from the company DOW CORNING such as DC200 of viscosity 60,000 cSt; and
the VISCASIL oils from GENERAL ELECTRIC and certain oils of the SF series (SF 96, SF 18) from GENERAL ELECTRIC.

Mention may also be made in a non-limiting manner of the polydimethylsiloxanes with dimethylsilanol end groups (Dimethiconol according to the CTFA designation) such as oils of the 48 series from the company RHONE POULENC.

Non-limiting mention may also be made of polydimethylsiloxanes with aminoethyl aminopropyl and alpha-omega silanol groups.

According to the present disclosure, in this class of polyalkylsiloxanes, suitable products include, but are not limited to those marketed under the designations “ABIL WAX 9800 and 9801” by the company GOLDSCHMIDT which are polyalkyl (C₁-C₂₀) siloxanes.

The polyalkylarylsiloxanes are chosen, for instance, from the linear and/or branched polydimethyl methylphenylsiloxanes, and polydimethyl diphenylsiloxanes with viscosity ranging from 1.10⁻⁵ to 5.10⁻² m²/s at 25° C.

Among those polyalkylarylsiloxanes, non-limiting mention may be made of the products marketed under the following designations:
the SILBIONE oils of series 70 641 from RHONE POULENC;
the oils of series RHODORSIL 70 633 and 763 from RHONE POULENC;
the oil DOW CORNING 556 COSMETIC GRAD FLUID from DOW CORNING;
silicones of the PK series from BAYER, such as the product PK20;
silicones of the series PN, PH from BAYER, such as the products PN1000 and PH1000; and
certain oils of the SF series from GENERAL ELECTRIC, such as SF 1023, SF 1154, SF 1250, SF 1265.

The silicone gums that can be present in the compositions according to the present disclosure are for example chosen from polydiorganosiloxanes with high number-average molecular weights ranging from 200,000 to 1,000,000, used alone or mixed with a solvent. This solvent can be chosen from volatile silicones, polydimethylsiloxane oils (PDMS), polyphenylmethylsiloxane oils (PPMS), isoparaffins, polyisobutylenes, methylene chloride, pentane, dodecane, tridecanes and mixtures thereof.

Non-limiting mention may be made of the following silicone gums:
polydimethylsiloxane gums,
polydimethylsiloxanes/methylvinylsiloxane gums,
polydimethylsiloxane/diphenylsiloxane gums,
polydimethylsiloxane/phenylmethylsiloxane gums, and
polydimethylsiloxane/diphenylsiloxane/methylvinylsiloxane gums.

According to at least one embodiment of the present disclosure, the products that can be used include, but are not limited to the following mixtures:
mixtures formed from a chain end hydroxylated polydimethylsiloxane (called dimethiconol according to the nomenclature of the CTFA dictionary) and a cyclic polydimethylsiloxane (called cyclomethicone according to the nomenclature of the CTFA dictionary) such as the product Q2 1401 marketed by the company DOW CORNING;
mixtures formed from a polydimethylsiloxane gum with a cyclic silicone such as the product SF 1214 Silicone Fluid from the company GENERAL ELECTRIC, this product is a gum SF 30 corresponding to a dimethicone, having a number-average molecular weight of 500,000 dissolved in oil SF 1202 Silicone Fluid corresponding to decamethylcyclopentasiloxane; and
mixtures of two PDMS of different viscosities, and more particularly of a PDMS gum and a PDMS oil, such as the product SF 1236 from the company GENERAL ELECTRIC. The product SF 1236 is a mixture of a gum SE 30 defined above having a viscosity of 20 m²/s and an oil SF 96 with a viscosity of 5.10⁻⁶ m²/s. According to at least one embodiment, this product comprises 15% of SE 30 gum and 85% of a SF 96 oil.

The organopolysiloxane resins optionally present in the compositions according to the present disclosure are, for example crosslinked siloxane systems containing the units: R₂SiO_{2/2}, R₃SiO_{1/2}, RSiO_{3/2} and SiO_{4/2} wherein R is chosen from C₁-C₁₆ hydrocarbon groups and phenyl groups. Among those products, non-limiting mention may be made of those wherein R is chosen from C₁-C₄ lower alkyl radicals, such as methyl and phenyl radicals.

Mention may also be made, in a non-limiting manner, of resins such as the product marketed under the designation “DOW CORNING 593” or those marketed under the designations “SILICONE FLUID SS 4230 and SS 4267” by the company GENERAL ELECTRIC, which are silicones of dimethyl/trimethyl siloxane structure.

Other suitable examples of resins include, but are not limited to trimethylsiloxysilicate resins, such as the product marketed under the designations X22-4914, X21-5034 and X21-5037 by the company SHIN-ETSU.

The organomodified silicones optionally present in the compositions according to the present disclosure are silicones as defined herein and include in their structure at least one organofunctional group fixed by a hydrocarbon radical.

Among the organomodified silicones, non-limiting mention may be made of the polyorganosiloxanes comprising:
polyethyleneoxy and/or polypropyleneoxy groups optionally comprising C₆-C₂₄ alkyl groups such as the products called dimethicone copolyol marketed by the company DOW CORNING under the designation DC 1248 or the oils SILWET L 722, L 7500, L 77, L 711 from the company UNION CARBIDE and the alkyl (C₁₂) methicone copolyol marketed by the company DOW CORNING under the designation Q2 5200;
thiol groups such as the products marketed under the designations “GP 72 A” and “GP 71” by GENESEE;
alkoxylated groups such as the product marketed under the designation “SILICONE COPOLYMER F-755” by SWS SILICONES and ABIL WAX 2428, 2434 and 2440 by the company GOLDSCHMIDT;
hydroxylated groups such as the polyorganosiloxanes with hydroxyalkyl function described in French Patent Application No. FR-A-85 16334;
acyloxyalkyl groups, for example the polyorganosiloxanes described in U.S. Pat. No. 4,957,732;
anionic groups of the carboxyl type, for example in the products described in European Patent No. 0 186 507 from the company CHISSO CORPORATION, or of the alkylcarboxyl type such as those present in the product X-22-3701 E from the company SHIN-ETSU; 2-hydroxyalkylsulphonate; 2-hydroxyalkylthiosulphate such as the products marketed by the company GOLDSCHMIDT under the designations “ABIL S201” and “ABIL S255”;
hydroxyacylamino groups, such as the polyorganosiloxanes described in European Patent Application No. 0 342 834. Mention may also be made of the product Q2-8413 from the company DOW CORNING.

Among the organomodified silicones, non-limiting mention may also be made of the aminated silicones.

As used herein, “aminated silicone” is understood to mean any silicone having at least one primary, secondary, tertiary amine function or a quaternary ammonium group.

The aminated silicones optionally used in the cosmetic compositions according to the present disclosure are chosen from:
(a) the compounds corresponding to the following formula (VI):

(R¹)ₐ(T)₃₋ₐ-Si[OSi(T)₂]ₙ-[OSi(T)_{b}(R¹)_{2-b}]ₘ—OSi(T)₃₋ₐ-(R¹)a (VI)
wherein

T is chosen from a hydrogen atom, phenyl, hydroxyl (—OH), and C₁-C₈ alkyl radicals, such as methyl, and C₁-C₈ alkoxy groups, such as methoxy,

a is an integer from 0 to 3, and in at least one embodiment, a is 0,

b is 0 or 1, and in at least one embodiment, b is 1,

m and n are numbers such that the sum (n+m) ranges from 1 to 2000, for example, ranges from 50 to 150; wherein n is a number ranging from 0 to 1999, such as ranging from 49 to 149, and m is a number ranging from 1 to 2000, such as ranging from 1 to 10;

R¹ is a monovalent radical of formula —C_{q}H_{2q}L wherein q is a number ranging from 2 to 8, and L is an amino group optionally quaternized, chosen from:

—N(R²)—CH₂—CH₂—N(R²)₂;

—N(R²)₂; —N⁺(R²)₃ Q-;

—N⁺(R²) (H)₂ Q-;

—N⁺(R²)₂HQ-; and

—N(R²)—CH₂—CH₂—N⁺R²)(H)₂ Q-,
wherein R² is chosen from a hydrogen atom, phenyl, benzyl, and saturated monovalent hydrocarbon radicals, for example C₁-C₂₀ alkyl radicals, and Q- is chosen from halide ions, for example fluoride, chloride, bromide and iodide.

According to at least one embodiment, the aminated silicones corresponding to the definition of formula (VI) are chosen from the compounds corresponding to the following formula:

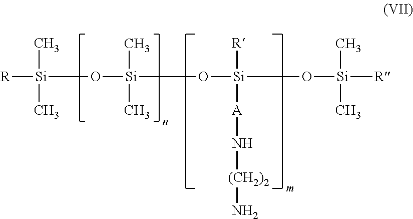

wherein R, R′, R″, are each independently chosen from C₁-C₄ alkyl radicals, such as CH₃; C₁-C₄ alkoxy radicals, for example methoxy; and OH; A is chosen from linear and branched C₃-C₈ alkylene radicals, such as C₃-C₆ alkylene radicals; and m and n are integers that depend on the molecular weight and whose sum ranges from 1 to 2000.

According to at least one embodiment of the present disclosure, R, R′, R″, are each independently chosen from C₁-C₄ alkyl radicals and hydroxyl radicals, A is a C₃ alkylene radical and m and n are such that the weight-average molecular weight of the compound ranges from 5,000 to 500,000. Compounds of this type are called “amodimethicone” in the CTFA dictionary.

According to another embodiment of the present disclosure, R, R′, R″, are each independently chosen from C₁-C₄ alkoxy radicals and hydroxyl radicals, wherein at least one of the radicals R or R″ is an alkoxy radical, and A is a C₃ alkylene radical. The molar ratio of hydroxy to alkoxy ranges from, for example 0.2/1 to 0.4/1, and in at least one embodiment, is equal to 0.3/1. Moreover, m and n are such that the weight-average molecular weight of the compound ranges from 2,000 to 10⁶, n ranges from 0 to 999, and m ranges from 1 to 1000, wherein the sum of n and m ranges from 1 to 1000.

Non-limiting examples of such compounds include, but are not limited to the product BELSIL® ADM 652, marketed by Wacker.

According to at least one embodiment of the present disclosure, R, R″, are each independently chosen from C₁-C₄ alkoxy radicals and hydroxyl radicals, wherein at least one of the radicals R, R″ is an alkoxy radical, R′ is a methyl radical and A is a C₃ alkylene radical. The molar ratio of hydroxy to alkoxy ranges from, for instance 1/0.8 to 1/1.1, and in at least one embodiment is equal to 1/0.95. Moreover, m and n are such that the weight-average molecular weight of the compound ranges from 2000 to 200,000. For instance, n ranges from 0 to 999 and m ranges from 1 to 1000, wherein the sum of n and m ranges from 1 to 1000.

Non-limiting examples of such compounds include, but are not limited to the product FLUIDWR® 1300, marketed by Wacker.

According to the present disclosure, the molecular weight of these silicones is determined by gel permeation chromatography (room temperature, polystyrene standard; μ styragem columns; eluent THF; flow of 1 mm/m; 200 μl of a 0.5 weight. % solution of silicone in THF is injected and detection is by refractometry and with a UV meter).

A product corresponding to the definition of formula (VI) is, for example the polymer called “trimethylsilylamodimethicone” in the CTFA dictionary, corresponding to the following formula (VIII):

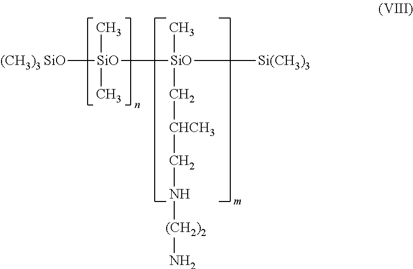

wherein n and m have the meanings given above according to formula (VI).

Such compounds are described, for instance in European Patent Application 0 095 238; a compound of formula (VIII) is for example sold under the designation Q2-8220 by the company OSI.
(b) the compounds corresponding to the following formula (IX):

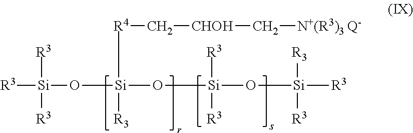

wherein,

R³ is chosen from monovalent C₁-C₁₈ hydrocarbon radicals, such as C₁-C₁₈ alkyl radicals and C₂-C₁₈ alkenyl radicals, for example methyl;

R⁴ is chosen from divalent hydrocarbon radicals, for instance C₁-C₁₈ alkylene radicals and divalent C₁-C₁₈ radicals, for example C₁-C₈ alkyleneoxy radical;

Q⁻ is a halide ion, and in at least one embodiment chloride;
r is a statistical average value ranging from 2 to 20, such as ranging from 2 to 8;
s is a statistical average value ranging from 20 to 200, for example ranging from 20 to 50.

Such compounds are described, for example in U.S. Pat. No. 4,185,087.

A non-limiting example of such a compound is the product sold by the company Union Carbide under the designation “UCAR SILICONE ALE 56”.
c) the quaternary ammonium silicones of formula (X):

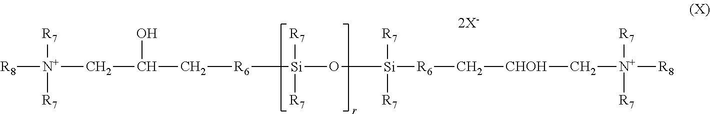

wherein:

Each instance of R₇ is independently chosen from monovalent C₁-C₁₈ hydrocarbon radicals, such as C₁-C₁₈ alkyl radicals, C₂-C₁₈ alkenyl radicals, and rings comprising 5 or 6 carbon atoms, for example methyl;

Each instance of R₆ is independently chosen from divalent hydrocarbon radicals, such as C₁-C₁₈ alkylene radicals and divalent C₁-C₁₈ radicals, for example C₁-C₈ alkyleneoxy radicals joined to the Si by an SiC bond;

Each instance of R₈ is independently chosen from a hydrogen atom, monovalent C₁-C₁₈ hydrocarbon radicals, such as C₁-C₁₈ alkyl radicals, C₂-C₁₈ alkenyl radicals, and —R₆—NHCOR₇ radicals;

X— is an anion such as a halide ion, for instance a chloride or a salt of an organic acid (acetate etc.);

r is a statistical average value ranging from 2 to 200, for example ranging from 5 to 100.

These silicones are, for example described in European Patent Application No. 0 530 974.
d) the aminated silicones of formula (XI):

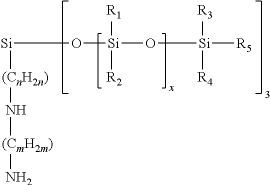

wherein:
R₁, R₂, R₃ and R₄, are each independently chosen from C₁-C₄ alkyl radicals and phenyl groups,
R₅ is chosen from C₁-C₄ alkyl radicals and hydroxyl groups,
n is an integer ranging from 1 to 5,
m is an integer ranging from 1 to 5,
and wherein x is chosen in such a way that the amine index ranges from 0.01 to 1 meq/g.

According to at least one embodiment of the present disclosure, the silicones are chosen from polydimethylsiloxanes, dimethicones and amodimethicones.

According to another embodiment of the present disclosure, the silicones chosen from polydimethylsiloxanes, dimethicones and amodimethicones are combined with at least one surfactant chosen from cationic and non-ionic surfactants.

For instance, non-limiting mention may be made of the product sold under the designation “EMULSION CATIONIQUE DC 939” by the company Dow Corning, which comprises, apart from amodimethicone, a cationic surfactant, namely trimethylcetylammonium chloride and a non-ionic surfactant of formula: C₁₃H₂₇—(OC₂H₄)₁₂—OH, known by the CTFA designation “trideceth-12”.

Another suitable commercial product according to the present disclosure is the product sold under the designation “DOW CORNING Q2 7224” by the company Dow Corning, comprising, in combination with the trimethylsilylamodimethicone of formula (VIII) described above, a non-ionic surfactant of formula: C₈H₁₇—C₆H₄—(OCH₂CH₂)₄₀—OH, known by the CTFA designation “octoxynol-40”, a second non-ionic surfactant of formula: C₁₂H₂₅—(OCH₂—CH₂)₆—OH, known by the CTFA designation “isolaureth-6”, and propylene glycol.

The at least one silicone is present in an amount ranging from 0.1 to 20 weightweight. %, such as ranging from 0.1 to 10 weight. %, relative to the total weight of the composition.

The compositions according to the present disclosure comprise a cosmetically acceptable medium.

This medium is, in at least one embodiment, aqueous, i.e. it comprises either only water, or water and at least one solvent, for example ethanol, propylene glycol, butylene glycol, isopropanol, glycol ethers such as the alkyl (C₁-C₄) ethers of mono-, di- or tripropylene glycol, butylene glycol, isopropanol, glycol ethers such as the alkyl (C₁-C₄) ethers of mono-, di- or tripropylene glycol, mono-, di- or triethylene glycol, dipropylene glycol, diethylene glycol and mixtures thereof.

The cosmetically acceptable medium can also be anhydrous or essentially anhydrous.

The cosmetically acceptable medium is aqueous in at least one embodiment of the present disclosure.

The compositions according to the present disclosure can further comprise at least one additive used in the field of application in question.

For example, the compositions disclosed herein can comprise perfumes, UV filters, preservatives, antioxidants, pH regulators, sequestering agents, anti-free-radical agents, hydrating agents, reducing agents, conditioners other than silicones and cationic surfactants such as esters, and vitamins.

The present disclosure further relates to the use of the composition as defined previously for conditioning the hair.

Notwithstanding that the numerical ranges and parameters setting forth the broad scope of the disclosure are approximations, unless otherwise indicated the numerical values set forth in the specific examples are reported as precisely as possible. Any numerical value, however, inherently contains certain errors necessarily resulting from the standard deviation found in their respective testing measurements.

The following examples serve to illustrate embodiments of the present disclosure without, however, exhibiting a limiting nature.

### EXAMPLES

### Example 1

A composition in accordance with the present disclosure was prepared as set forth in Table 1 below. The contents are expressed in g of active substances per 100 g of composition.

TABLE 1

| Perfume | 0.4 | g |
|---|---|---|
| Caprylyl Glycol | 0.2 | g |
| Yellow 5 | 0.003 | g |
| Hollow microspheres of vinylidene | 5.6 | g |
| chloride/acrylonitrile/methyl methacrylate copolymer | | |
| expanded with isobutane (EXPANCEL 551 DE 40 | | |
| d42 from the company Expancel) | | |
| Red 4 | 0.003 | g |
| Benzoic acid | 0.2 | g |
| Isopropyl alcohol | 0.5 | g |
| Cetearyl alcohol | 3 | g |
| Behenyl trimethyl ammonium chloride | 2.4 | g |
| 96/4 mixture of sodium salt of potato | 7.0 | g |
| carboxymethylstarch and ethanol (PRIMOJEL from | | |
| the company DMV International) | | |
| Citric acid | Qs pH 4 | |
| Water | Q.s. 100 | g |

### Example 2

A composition in accordance with the present disclosure was prepared as set forth in Table 2 below. The contents are expressed in g of active substances per 100 g of composition.

TABLE 2

| Perfume | 0.4 | g |
|---|---|---|
| Caprylyl Glycol | 0.2 | g |
| Yellow 5 | 0.003 | g |
| Hollow microspheres of vinylidene | 5.1 | g |
| chloride/acrylonitrile/methyl methacrylate copolymer | | |
| expanded with isobutane (EXPANCEL 551 DE 40 d42 | | |
| from the company Expancel) | | |
| Red 4 | 0.003 | g |
| Benzoic acid | 0.2 | g |
| Cetearyl alcohol | 1.5 | g |
| 96/4 mixture of sodium salt of potato | 4.4 | g |
| carboxymethylstarch and ethanol (PRIMOJEL from | | |
| the company DMV International) | | |
| Distearoylethyl hydroxyethylammonium methosulphate | 4.5 | g |
| Citric acid | Qs pH 4 | |
| Water | Q.s. 100 | g |

### Example 3

A composition in accordance with the present disclosure was prepared as set forth in Table 3 below. The contents are expressed in g of active substances per 100 g of composition.

TABLE 3

| Perfume | 0.4 | g |
|---|---|---|
| Caprylyl Glycol | 0.2 | g |
| Yellow 5 | 0.003 | g |
| Hollow microspheres of vinylidene | 5.1 | g |
| chloride/acrylonitrile/methyl methacrylate copolymer | | |
| expanded with isobutane (EXPANCEL 551 DE 40 d42 | | |
| from the company Expancel) | | |
| Red 4 | 0.003 | g |
| Benzoic acid | 0.2 | g |
| Cetearyl alcohol | 4.5 | g |
| 96/4 mixture of sodium salt of potato | 4.4 | g |
| carboxymethylstarch and ethanol (PRIMOJEL) from | | |
| the company DMV International) | | |
| Distearoylethyl hydroxyethylammonium methosulphate | 4.5 | g |
| Citric acid | Qs pH 4 | |
| Water | Q.s. 100 | g |

### Example 4

A composition in accordance with the present disclosure was prepared as set forth in Table 4 below. The contents are expressed in g of active substances per 100 g of composition.

TABLE 4

| Perfume | 0.4 | g |
|---|---|---|
| Caprylyl Glycol | 0.2 | g |
| Yellow 5 | 0.003 | g |
| Hollow microspheres of vinylidene | 4.7 | g |
| chloride/acrylonitrile/methyl methacrylate copolymer | | |
| expanded with isobutane (EXPANCEL 551 DE 40 d42 | | |
| from the company Expancel) | | |
| Red 4 | 0.003 | g |
| Benzoic acid | 0.2 | g |
| Cetearyl alcohol | 4.2 | g |
| Jojoba seed oil (PURCELL JOJOBA INT) | 1.4 | g |
| 96/4 mixture of sodium salt of potato | 7.0 | g |
| carboxymethylstarch and ethanol (PRIMOJEL) from | | |
| the company DMV International) | | |
| Distearoylethyl hydroxyethylammonium methosulphate | 4.2 | g |
| Citric acid | Qs pH 4 | |
| Water | Q.s. 100 | g |

### Example 5

A composition in accordance with the present disclosure was prepared as set forth in Table 5 below. The contents are expressed in g of active substances per 100 g of composition.

TABLE 5

| Perfume | 0.4 | g |
|---|---|---|
| Caprylyl Glycol | 0.2 | g |
| Cetyl trimethylammonium chloride | 0.03 | g |
| Yellow 5 | 0.003 | g |
| Hollow microspheres of vinylidene | 5.1 | g |
| chloride/acrylonitrile/methyl methacrylate copolymer | | |
| expanded with isobutane (EXPANCEL 551 DE 40 d42 | | |
| from the company Expancel) | | |
| Red 4 | 0.003 | g |
| Benzoic acid | 0.2 | g |
| Cetearyl alcohol | 4.5 | g |
| Amodimethicone in emulsion at 60% active substance | 1.7 | g |
| (DOW CORNING 939 emulsion from Dow Corning) | | |
| Trideceth-6 | 0.1 4 | g |
| 96/4 mixture of sodium salt of potato | 6.4 | g |
| carboxymethylstarch and ethanol (PRIMOJEL from | | |
| the company DMV International) | | |
| Distearoylethyl hydroxyethylammonium | 4.6 | g |
| methosulphate | | |
| Citric acid | Qs pH 4 | |
| Water | Q.s. 100 | g |

### Example 6

A composition in accordance with the present disclosure was prepared as set forth in Table 6 below. The contents are expressed in g of active substances per 100 g of composition.

TABLE 6

| Perfume | 0.4 | g |
|---|---|---|
| Caprylyl Glycol | 0.2 | g |
| 76/24 mixture of dimethicone and crosslinked | 3.1 | g |
| dimethicone/vinyldimethicone copolymer (KSG 16 | | |
| from the company Shin Etsu) | | |
| Yellow 5 | 0.003 | g |
| Hollow microspheres of vinylidene | 3.4 | g |
| chloride/acrylonitrile/methyl methacrylate copolymer | | |
| expanded with isobutane (EXPANCEL 551 DE 40 d42 | | |
| from the company Expancel) | | |
| Red 4 | 0.003 | g |
| Benzoic acid | 0.2 | g |
| Cetearyl alcohol | 4.7 | g |
| 96/4 mixture of sodium salt of potato | 5.5 | g |
| carboxymethylstarch and ethanol (PRIMOJEL from | | |
| the company DMV International) | | |
| Distearoylethyl hydroxyethylammonium | 4.7 | g |
| methosulphate | | |
| Citric acid | Qs pH 4 | |
| Water | Q.s. 100 | g |

### Example 7

A composition in accordance with the present disclosure was prepared as set forth in Table 7 below. The contents are expressed in g of active substances per 100 g of composition.

TABLE 7

| Perfume | 0.4 | |
|---|---|---|
| Caprylyl Glycol | 0.2 | |
| Yellow 5 | 0.003 | |
| Hollow microspheres of vinylidene | 5.6 | g |
| chloride/acrylonitrile/methyl methacrylate copolymer | | |
| expanded with isobutane (EXPANCEL 551 DE 40 d42 | | |
| from the company Expancel) | | |
| Red 4 | 0.003 | g |
| Benzoic acid | 0.2 | g |
| Isopropyl alcohol | 0.5 | g |
| Cetearyl alcohol | 3 | g |
| Behenyl trimethyl ammonium chloride | 2.4 | g |
| 96/4 mixture of sodium salt of potato | 7.0 | g |
| carboxymethylstarch and ethanol (PRIMOJEL from | | |
| the company DMV International) | | |
| 3-aminopropyl triethoxysilane | 3 | g |
| Citric acid | Qs pH 4 | |
| Water | Q.s. 100 | g |

### Example 8

A composition in accordance with the present disclosure was prepared as set forth in Table 8 below. The contents are expressed in g of active substances per 100 g of composition.

TABLE 8

| Perfume | 0.4 | g |
|---|---|---|
| Caprylyl Glycol | 0.2 | g |
| Yellow 5 | 0.003 | g |
| Hollow microspheres of vinylidene | 5.6 | g |
| chloride/acrylonitrile/methyl methacrylate copolymer | | g |
| expanded with isobutane (EXPANCEL 551 DE 40 d42 | | |
| from the company Expancel) | | |
| Red 4 | 0.003 | g |
| Citric acid | 0 | g |
| Benzoic acid | 0.2 | g |
| Isopropyl alcohol | 0.5 | g |
| Cetearyl alcohol | 3 | g |
| Behenyl trimethyl ammonium chloride | 2.4 | g |
| 96/4 mixture of sodium salt of potato | 7.0 | g |
| carboxymethylstarch and ethanol (PRIMOJEL from | | |
| the company DMV International) | | |
| Spruce sawdust (extracts of Picea excelsa) (wood | 3 | g |
| flour F140 from SPPS) | | |
| Citric acid | Qs pH 4 | |
| Water | Q.s. 100 | g |

### Example 9

A composition in accordance with the present disclosure was prepared as set forth in Table 9 below. The contents are expressed in g of active substances per 100 g of composition.

TABLE 9

| Perfume | 0.4 | g |
|---|---|---|
| Caprylyl Glycol | 0.2 | g |
| Yellow 5 | 0.003 | g |
| Hollow microspheres of vinylidene | 5.1 | g |
| chloride/acrylonitrile/methyl methacrylate copolymer | | |
| expanded with isobutane (EXPANCEL 551 DE 40 d42 | | |
| from the company Expancel) | | |
| Red 4 | 0.003 | g |
| Benzoic acid | 0.2 | g |
| Cetearyl alcohol | 1.5 | g |
| 96/4 mixture of sodium salt of potato | 4.4 | g |
| carboxymethylstarch and ethanol (PRIMOJEL from | | |
| the company DMV International) | | |
| Distearoylethyl hydroxyethylammonium methosulphate | 4.5 | g |
| Spruce sawdust (extracts of Picea excelsa) (wood | 1.9 | g |
| flour F140 from SPPS) | | |
| Citric acid | Qs pH 4 | |
| Water | Q.s. 100 | g |

### Example 10

A composition in accordance with the present disclosure was prepared as set forth in Table 10 below. The contents are expressed in percentages by weight g of active substances per 100 g of composition.

TABLE 10

| Perfume | 0.4 | g |
|---|---|---|
| Caprylyl Glycol | 0.2 | g |
| Yellow 5 | 0.003 | g |
| Hollow microspheres of vinylidene | 5.1 | g |
| chloride/acrylonitrile/methyl methacrylate copolymer | | |
| expanded with isobutane (EXPANCEL 551 DE 40 d42 | | |
| from the company Expancel) | | |
| Red 4 | 0.003 | g |
| Benzoic acid | 0.2 | g |
| Cetearyl alcohol | 1.5 | g |
| Powdered seeds of Prunus armenica (Apricot) | 10 | g |
| 96/4 mixture of sodium salt of potato | 4.4 | g |
| carboxymethylstarch and ethanol (PRIMOJEL from | | |
| the company DMV International) | | |
| Distearoylethyl hydroxyethylammonium methosulphate | 4.5 | g |
| Citric acid | Qs pH 4 | |
| Water | Q.s. 100 | g |

### Example 11

A composition in accordance with the present disclosure was prepared as set forth in Table 11 below. The contents are expressed in g of active substances per 100 g of composition.

TABLE 11

| Perfume | 0.4 | g |
|---|---|---|
| Caprylyl Glycol | 0.2 | g |
| Yellow 5 | 0.003 | g |
| Hollow microspheres of vinylidene | 5.1 | g |
| chloride/acrylonitrile/methyl methacrylate copolymer | | |
| expanded with isobutane (EXPANCEL 551 DE 40 d42 | | |
| from the company Expancel) | | |
| Red 4 | 0.003 | g |
| Benzoic acid | 0.2 | g |
| Cetearyl alcohol | 4.3 | g |
| 96/4 mixture of sodium salt of potato | 7.0 | g |
| carboxymethylstarch and ethanol (PRIMOJEL from | | |
| the company DMV International) | | |
| Distearoylethyl hydroxyethylammonium methosulphate | 4.3 | g |
| Spruce sawdust (extracts of Picea excelsa) (wood | 3 | g |
| flour F140 from SPPS) | | |
| Citric acid | Qs pH 4 | |
| Water | Q.s. 100 | g |

### Example 12

A composition in accordance with the present disclosure was prepared as set forth in Table 12 below. The contents are expressed in g of active substances per 100 g of composition.

TABLE 12

| Perfume | 0.4 | g |
|---|---|---|
| Caprylyl Glycol | 0.2 | g |
| Yellow 5 | 0.003 | g |
| Hollow microspheres of vinylidene | 5.1 | g |
| chloride/acrylonitrile/methyl methacrylate copolymer | | |
| expanded with isobutane (Expancel 551 DE 40 d42 | | |
| from the company Expancel) | | |
| Red 4 | 0.003 | g |
| Benzoic acid | 0.2 | g |
| Cetearyl alcohol | 4.4 | g |
| Jojoba seed oil | 1.5 | g |
| 96/4 mixture of sodium salt of potato | 4.4 | g |
| carboxymethylstarch and ethanol (PRIMOJEL from | | |
| the company DMV International) | | |
| Distearoylethyl hydroxyethylammonium methosulphate | 4.4 | g |
| Spruce sawdust (extracts of Picea excelsa) (wood | 1.9 | g |
| flour F140 from SPPS) | | |
| Citric acid | Qs pH 4 | |
| Water | Q.s. 100 | g |

### Example 13

A composition in accordance with the present disclosure was prepared as set forth in Table 13 below. The contents are expressed in g of active substances per 100 g of composition.

TABLE 13

| Perfume | 0.4 | g |
|---|---|---|
| Caprylyl Glycol | 0.2 | g |
| Cetyl trimethylammonium chloride | 0.03 | g |
| Yellow 5 | 0.003 | g |
| Hollow microspheres of vinylidene | 5.1 | g |
| chloride/acrylonitrile/methyl methacrylate copolymer | | |
| expanded with isobutane (EXPANCEL 551 DE 40 d42 | | |
| from the company Expancel) | | |
| Red 4 | 0.003 | g |
| Benzoic acid | 0.2 | g |
| Cetearyl alcohol | 4.5 | g |
| Amodimethicone | 1.7 | g |
| Tridiceth-6 | 0.2 | g |
| 96/4 mixture of sodium salt of potato | 6.4 | g |
| carboxymethylstarch and ethanol (PRIMOJEL from | | |
| the company DMV International) | | |
| Distearoylethyl hydroxyethylammonium methosulphate | 4.6 | g |
| Spruce sawdust (extracts of Picea excelsa) | 2.7 | g |
| Citric acid | Qs pH 4 | |
| Water | Q.s. 100 | g |

### Example 14

A composition in accordance with the present disclosure was prepared as set forth in Table 14 below. The contents are expressed in g of active substances per 100 g of composition.

TABLE 14

| Perfume | 0.4 | g |
|---|---|---|
| Caprylyl Glycol | 0.2 | g |
| 76/24 mixture of dimethicone and of crosslinked | 3.1 | g |
| dimethicone/vinyldimethicone copolymer (KSG 16 | | |
| from the company Shin Etsu) | | |
| Yellow 5 | 0.003 | g |
| Hollow microspheres of vinylidene | 3.4 | g |
| chloride/acrylonitrile/methyl methacrylate copolymer | | |
| expanded with isobutane (EXPANCEL 551 DE 40 d42 | | |
| from the company Expancel) | | |
| Red 4 | 0.003 | g |
| Benzoic acid | 0.2 | g |
| Cetearyl alcohol | 4.7 | g |
| 96/4 mixture of sodium salt of potato | 5.5 | g |
| carboxymethylstarch and ethanol (PRIMOJEL from | | |
| the company DMV International) | | |
| Distearoylethyl hydroxyethylammonium | 4.7 | g |
| methosulphate | | |
| Spruce sawdust (extracts of Picea excelsa) | 2.34 | g |
| Citric acid | Qs pH 4 | |
| Water | Q.s. 100 | g |

### Example 15

A composition in accordance with the present disclosure was prepared as set forth in Table 15 below. The contents are expressed in g of active substances per 100 g of composition.

TABLE 15

| Perfume | 0.4 | g |
|---|---|---|
| Caprylyl Glycol | 0.2 | g |
| Yellow 5 | 0.003 | g |
| Hollow microspheres of vinylidene | 5.6 | g |
| chloride/acrylonitrile/methyl methacrylate copolymer | | |
| expanded with isobutane (EXPANCEL 551 DE 40 d42 | | |
| from the company Expancel) | | |
| Red 4 | 0.003 | g |
| Benzoic acid | 0.2 | g |
| Isopropyl alcohol | 0.5 | g |
| Cetearyl alcohol | 3 | g |
| Behenyl trimethyl ammonium chloride | 2.3 | g |
| 96/4 mixture of sodium salt of potato | 7.0 | g |
| carboxymethylstarch and ethanol (PRIMOJEL from | | |
| the company DMV International) | | |
| Spruce sawdust (extracts of Picea excelsa) | 3 | g |
| Aminopropyl triethoxysilane | 3 | g |
| Citric acid | Qs pH 4 | |
| Water | Q.s. 100 | g |

The compositions in the form of deformable solids according to examples 1 to 15 are hair care products which had very good cosmetic performance by making disentangling very easy and imparting great softness to the hair, including normal to dry hair. A good styling effect was also noted. The qualities of use were good: a fairly small amount of product by weight was generally sufficient for treating all the hair. It was simple to apply to the hair, and rinsing was very easy and quick.

### Example 16

A composition in accordance with the present disclosure was prepared (composition B) having a weight ratio of at least one cationic surfactant to at least one starch greater than 0.3. Composition B was compared with a comparative composition (composition A) having a weight ratio of at least one cationic surfactant to at least one starch less than 0.3.

Composition B according to the present disclosure and composition A outside of the scope of the claims were prepared in accordance with the formulations given in Table 16 below. The contents are expressed in g of active substances per 100 g of composition.

TABLE 16

| | | B (according |
|---|---|---|
| | A | to the present |
| | (comparative) | disclosure) |
| Crosslinked potato carboxymethylstarch | 27 | 20.7 |
| (PRIMOJEL from the company DMV | | |
| International) | | |
| Spruce wood flour (WOOD FLOUR | 22 | 22 |
| F140 from SPPS) | | |
| Expanded microspheres of | 5 | 5 |
| acrylonitrile/methyl methacrylate | | |
| copolymer (EXPANCEL 551 DE 40 | | |
| d42 from AKZO NOBEL) | | |
| Propylene glycol | 16 | 16 |
| Cetyl trimethylammonium chloride | 4 | 10.3 |
| (GENAMIN CTAC 50 ET from the | | |
| company CLARIANT) | | |
| Dimethyldiallylammonium chloride/ | 2.7 | 2.7 |
| acrylic acid copolymer (MERQUAT | | |
| 280 from the company NALCO) | | |
| Water | Qs 100 | Qs 100 |
| Cationic surfactant/starch ratio | 0.15 | 0.5 |

The texture of composition A was very dry and very friable. In water, it disintegrated moderately well (clusters remained in the hands), and resulted in a texture that was not very smooth after being converted to cream in the hand.

Composition B was less dry and less friable. It disintegrated substantially better than composition A, and resulted in a smoother texture after being converted to cream in the hand.

Compositions A and B were applied per half-head on a styling head, in an amount of 6 g of composition per half-head, then rinsed with water.

7 experts evaluated the properties on a scale of 0 (not good) to 5 (very good).

Table 17 collates the averages of the grades obtained over the criteria evaluated.

TABLE 17

| | | B (according |
|---|---|---|
| | A | to the present |
| | (comparative) | disclosure) |
| Melting on application | 0.8 | 2.1 |
| Suppleness of the hair on | 1.1 | 2.4 |
| application | | |
| Suppleness of the hair on rinsing | 0.8 | 2.2 |
| Disentangling of wet hair | 1.5 | 2.5 |
| Suppleness of wet hair | 1.2 | 2.1 |

Composition B in accordance with the present disclosure resulted in cosmetic property scores that were substantially higher than those of composition A.

### Example 17

A composition in accordance with the present disclosure was prepared (composition C) having a weight ratio of at least one cationic surfactant to at least one starch greater than 0.3. Composition C was compared with the comparative composition A from Example 16 having a weight ratio of at least one cationic surfactant to at least one starch less than 0.3.

Composition C according to the present disclosure and composition A outside of the scope of the claims were prepared in accordance with the formulations given in Table 18. The contents are expressed in g of active substances per 100 g of composition.

TABLE 18

| | | C (according |
|---|---|---|
| | A | to the present |
| | (comparative) | disclosure) |
| Crosslinked potato carboxymethylstarch | 27 | 8 |
| (PRIMOJEL from the company DMV | | |
| International) | | |
| Spruce wood flour (WOOD FLOUR | 22 | 22 |
| F140 from SPPS) | | |
| Expanded microspheres of | 5 | 5 |
| acrylonitrile/methyl methacrylate | | |
| copolymer (EXPANCEL 551 DE 40 | | |
| d42 from AKZO NOBEL) | | |
| Propylene glycol | 16 | 16 |
| Cetyl trimethylammonium chloride | 4 | 4 |
| (GENAMIN CTAC 50 ET from the | | |
| company CLARIANT) | | |
| Dimethyldiallylammonium chloride/ | 2.7 | 2.7 |
| acrylic acid copolymer (MERQUAT | | |
| 280 from the company NALCO) | | |
| Water | Qs 100 | Qs 100 |
| Cationic surfactant/starch ratio | 0.15 | 0.5 |

The texture of composition A was very dry and very friable. In water, it disintegrated moderately well (clusters remained in the hands), and resulted in a texture that was not very smooth after being converted to cream in the hand.

Composition C was not dry and was not friable. It exhibited very good disintegration and resulted in a very smooth texture after being converted to cream in the hand.

Compositions A and C were applied per half-head on a styling head, in an amount of 6 g of composition per half-head, then were rinsed with water.

6 experts evaluated the properties on a scale of 0 (not good) to 5 (very good).

Table 19 collates the averages of the grades obtained over the criteria evaluated.

TABLE 19

| | | C (according |
|---|---|---|
| | A | to the present |
| | (comparative) | disclosure) |
| Melting on application | 0.8 | 2 |
| Smooth feel on dry hair | 2.5 | 3 |

Composition C in accordance with the present disclosure resulted in substantially higher cosmetic property scores than composition A.

What is claimed is:

1. A cosmetic composition comprising, in a cosmetically acceptable medium, at least one type of hollow particle, at least one starch chosen from modified and unmodified starches, and at least one cationic surfactant, wherein the weight ratio of the at least one cationic surfactant to the at least one starch has a value greater than or equal to 0.3; and
wherein the cosmetic composition is in the form of a deformable solid.

2. The cosmetic composition according to claim 1, wherein the weight ratio of the at least one cationic surfactant to the at least one starch has a value ranging from 0.3 to 10.

3. The cosmetic composition according to claim 1, wherein the at least one type of hollow particle is chosen from glass particles; particles of thermoplastic materials chosen from polyamides, polymers and copolymers of acrylonitrile, particles of vinylidene chloride, particles of vinyl chloride, particles of acrylic monomer, particles of styrene monomer, all optionally expanded; microporous microspheres, and silica microparticles.

4. The cosmetic composition according to claim 3, wherein the at least one type of hollow particle is chosen from hollow particles of an expanded copolymer of vinylidene chloride and acrylonitrile, and particles of vinylidene chloride, acrylonitrile and methyl methacrylate.

5. The cosmetic composition according to claim 1, wherein the at least one type of hollow particle is present in an amount ranging from 0.01 to 15 weight %, relative to the total weight of the composition.

6. The cosmetic composition according to claim 1, wherein the at least one starch is chosen from at least one vegetable source chosen from maize, peas, potato, sweet potato, banana, barley, wheat, rice, oat, sago, tapioca and sorghum.

7. The cosmetic composition according to claim 1, wherein the at least one starch is chosen from hydroxypropylated distarch phosphate, starch modified with 2-chloroethylaminodipropionic acid and carboxymethylstarches.

8. The cosmetic composition according to claim 1, wherein the at least one starch is present in an amount ranging from 0.01 to 15 weight %, of the total weight of the composition.

9. The cosmetic composition according to claim 1, wherein the at least one cationic surfactant is present in an amount ranging from 0.1 to 10 weight % relative to the total weight of the composition.

10. The cosmetic composition according to claim 1, further comprising at least one additional particulate agent other than the at least one type of hollow particle.

11. The cosmetic composition according to claim 10, wherein the at least one additional particulate agent is chosen from vegetable powders, pigments and anti-dandruff agents.

12. The cosmetic composition according to claim 10, wherein the at least one additional particulate agent is present in an amount ranging from 0.01 to 30 weight % relative to the total weight of the composition.

13. The cosmetic composition according to claim 1, further comprising at least one non-silicone fat.

14. The cosmetic composition according to claim 1, further comprising at least one silicone.

15. The cosmetic composition according to claim 2, wherein the weight ratio of the at least one cationic surfactant to the at least one starch has a value ranging from 0.35 to 1.

16. The cosmetic composition according to claim 5, wherein the at least one type of hollow particle is present in an amount ranging from 1 to 10 weight %, relative to the total weight of the composition.

17. The cosmetic composition according to claim 8, wherein the at least one starch is present in an amount ranging from 1 to 10 weight %, of the total weight of the composition.

18. A method of conditioning the hair comprising:
applying to the hair a cosmetic composition comprising, in a cosmetically acceptable medium, at least one type of hollow particle, at least one starch chosen from modified and unmodified starches, and at least one cationic surfactant, wherein the weight ratio of the at least one cationic surfactant to the at least one starch has a value greater than or equal to 0.3; and wherein the cosmetic composition is in the form of a deformable solid; and
optionally rinsing the hair.
